(19) United States
(12) Patent Application Publication
(10) Pub. No.: US 2026/0294420 A1
(43) Pub. Date: Oct. 1, 2026

(54) MULTIFUNCTIONAL DETACHABLE ORAL RETRACTOR FOR ORTHODONTIC TREATMENT

(71) Applicant: Union Hospital, Tongji Medical College, Huazhong University of Science and Technology, Wuhan (CN)

(72) Inventors: Mengru Xie, Wuhan (CN); Jun Han, Wuhan (CN); Li Hu, Wuhan (CN); Xinlong Yang, Wuhan (CN)

(21) Appl. No.: 19/330,808

(22) Filed: Sep. 16, 2025

(30) Foreign Application Priority Data
Mar. 26, 2025 (CN) 202510363525.X

(51) Int. Cl. A61B 17/02; A61B 17/00

(52) U.S. Cl. CPC A61B 17/0206; A61B 2017/00455; A61B 2017/00544

(57) ABSTRACT

A multifunctional detachable oral retractor for orthodontic treatment includes a base body, middle parts of top and bottom ends of the base body are hinged with rotating arms, end portions of the rotating arms are hinged with a suspension frame, the end portions of the rotating arms are hinged with a forceps arm located on an outer side of the suspension frame, a shaft tube is slidably mounted on an end face of one side of the base body, a piston is slidably mounted inside the base body, and a pilot valve is mounted at a middle part of an end face of the other side of the base body. A physician can more intuitively sense and control a support force applied to a patient's oral cavity, ensuring reliable support while avoiding strain on perioral muscles, significantly enhancing patient comfort through a gentler support process.

### CROSS-REFERENCE TO RELATED APPLICATION

This application claims priority of Chinese Patent Application No. 202510363525.X, filed on Mar 26, 2025, the entire contents of which are incorporated herein by reference.

### TECHNICAL FIELD

The present disclosure relates to the technical field of oral retractors, and specifically to a multifunctional detachable oral retractor for orthodontic treatment.

### BACKGROUND

An oral retractor, also known as a mouth opener, is designed to maintain a patient's oral cavity in an open state, enlarge an operational space, and facilitate various dental operations for physicians. A Chinese patent provides an adjustable oral orthodontic retractor with application number 202322497536.6. This device is capable of adjusting the oral retractor's sizes to accommodate patients with varying oral cavity dimensions, while integrating an auxiliary lighting system that significantly enhances operational convenience for medical staff.

However, current oral retractors are not only constrained by unitary dimensional configurations and a singular expansion modality, failing to effectively accommodate individual variations in patients' oral cavity dimensions, but also often provide non-adjustable expansion ranges and support forces that rely on rigid mechanical adjustment mechanisms. Physicians lack intuitive and precise control over adjustments of expansion ranges and support forces, resulting in insufficient patient comfort and a risk of perioral muscle strain. Furthermore, the support provided is often unreliable, and the device cannot be utilized in subsequent rehabilitation training, thereby limiting its scope of application.

### SUMMARY

The present disclosure provides a multifunctional detachable oral retractor for orthodontic treatment, which effectively solves the problems existing in the background. Current oral retractors are not only constrained by unitary dimensional configurations and a singular expansion modality, failing to effectively accommodate individual variations in patients' oral cavity dimensions, but also often provide non-adjustable expansion ranges and support forces that rely on rigid mechanical adjustment mechanisms. Physicians lack intuitive and precise control over adjustments of expansion ranges and support forces, resulting in insufficient patient comfort and a risk of perioral muscle strain. Furthermore, the support provided is often unreliable, and the device cannot be utilized in subsequent rehabilitation training, thereby limiting its scope of application.

To realize the above objective, the present disclosure provides the following technical solutions: a multifunctional detachable oral retractor for orthodontic treatment includes a base body 1, middle parts of top and bottom ends of the base body are hinged with rotating arms, end portions of the rotating arms are hinged with a suspension frame, the end portions of the rotating arms are hinged with a forceps arm located on an outer side of the suspension frame, and a synchronous expansion mechanism is mounted on an end face of the suspension frame;

the synchronous expansion mechanism includes a shaft tube;

the shaft tube is slidably mounted on an end face of one side of the base body, a piston is slidably mounted inside the base body, a pilot valve is mounted at a middle part of an end face of the other side of the base body, a pressure gauge is mounted on one side of the top end of the base body, a conduit is mounted at an end portion of the shaft tube, a plug block is slidably mounted at one end of the forceps arm, an air cavity is disposed inside the forceps arm corresponding to the plug block, a finger pressure head is mounted at a middle part of a side end face of the plug block, a plug is slidably mounted on a side end face of the air cavity close to the plug block, and a spring is mounted at an end portion of the plug;

a sliding cylinder is mounted at the top end of the base body, straight tubes are slidably mounted at two ends of the sliding cylinder, plug rings are mounted at end portions of the straight tubes located inside the sliding cylinder, guide holes are disposed at middle parts of side end faces of the sliding cylinder and the piston, strip boxes are mounted at the other ends of the straight tubes, insertion tubes are threadedly mounted on the other sides of bottom ends of the strip boxes, and flaring seats are rotatably mounted at end portions of the insertion tubes; and

top boxes are mounted on two sides of one of the flaring seats, bottom boxes are mounted on two sides of the other flaring seat, ring plugs are slidably mounted inside the top boxes and the bottom boxes, cross tubes are mounted at end portions of the ring plugs, a vertical tube is mounted on one side of a bottom end of each top box, a cylinder body is mounted on one side of a top end of each bottom box, and a sealing ring is mounted at an end portion of the vertical tube located inside the cylinder body.

Preferably, the pilot valve is a one-way valve, the air cavity is communicated with the shaft tube through the conduit, and the shaft tube and the sliding cylinder are communicated with an inner cavity of the base body through the guide holes; and the plug has an I-shaped cross-sectional profile, and an original length of the spring is greater than a thickness of the air cavity.

Preferably, cheek plates are mounted at corners of arched portions of the top boxes, and the arched portions of the top boxes and the bottom boxes, along with the cheek plates, are designed to conform to a natural curvature of the human oral cavity.

Preferably, the top boxes and the bottom boxes are sealed and slidably connected to the cross tubes, and end portions of the cross tubes are threadedly connected to the flaring seats.

Preferably, interiors of the top boxes and the bottom boxes located on outer sides of the cross tubes, and interiors of the cylinder bodies located on outer sides of the vertical tubes, are all filled with clean air.

Preferably, the sealing ring is slidably connected to the cylinder body, the cylinder body is designed to conform to a shape of the sealing ring, and a sliding distance of the sealing ring is equal to that of the plug ring.

Preferably, valve housings are mounted on outer curved faces of the cross tubes close to the flaring seats, and drive bolts are threadedly mounted at tops of outer curved faces of the valve housings; a plug disk is mounted at an end portion of each drive bolt, and a cylindrical cavity is disposed inside each cross tube corresponding to the plug disk; and a plurality of side holes are disposed circumferentially at equal angles on the outer curved faces of the cross tubes located inside each flaring seat, flow-stop plates are slidably mounted inside the flaring seat in a symmetrical manner, a compression spring is mounted at a middle part of side end faces of the flow-stop plates, and a plurality of grooves are disposed circumferentially at equal angles on an outer curved face of each flow-stop plate;

a strip plate is slidably mounted inside each strip box, an ejector pin is mounted on an end face of the strip plate corresponding to the insertion tube, a flower-shaped plate is slidably mounted inside the insertion tube, and a plurality of protruding blocks are mounted circumferentially at equal angles at an end portion of the insertion tube; and lead screws are threadedly mounted on end faces of the flaring seats close to one side of the base body, toothed plates are mounted at end portions of the lead screws, and a clamping tube is clamped into a middle part of a top end of one of the toothed plates; and

a negative pressure bulb is mounted at one end of the clamping tube, a squeeze bulb is mounted at the other end of the negative pressure bulb, and check valves are mounted at two ends of the squeeze bulb corresponding to the clamping tube; connecting tubes are mounted on two sides of top ends of the top boxes, two sides of bottom ends of the bottom boxes, and two sides of outer curved faces of each cylinder body; and flat boxes are mounted at end portions of the connecting tubes, and sealing bolts are threadedly mounted on end faces of the flat boxes corresponding to the connecting tubes.

Preferably, an original length of the compression spring exceeds twice a sliding distance of the flow-stop plate, a minimum clearance between the flow-stop plate and the end face of the flaring seat is less than a distance between the valve housing and the side holes, a clearance between the strip plate and a bottom end of the strip box is less than a length of the insertion tube, and a minimum clearance between the flower-shaped plate and the end portion of the insertion tube is less than a length of the ejector pin; and an air valve is mounted on an end face of the strip box away from one side corresponding to a length of the insertion tube, and an interior of the strip box located between the air valve and the strip plate is filled with clean air.

Preferably, a toothed pad is adhesively bonded to an end face of each toothed plate, and a cross-sectional profile of the toothed pad is designed to conform to a dental arch curvature of the human oral cavity; and the check valves at the two ends of the squeeze bulb are an inlet one-way valve and an outlet one-way valve, the squeeze bulb is connected to the clamping tube via the inlet one-way valve, and the negative pressure bulb is a hollow mesh structure.

Preferably, spaces on two sides of each ring plug within an inner cavity of the top box or the bottom box, are interconnected via the connecting tube and the flat box; spaces on two sides of each sealing ring within an inner cavity of the cylinder body, are interconnected via the connecting tube and the flat box; and a length of the sealing bolt exceeds a thickness of the flat box, and an end portion of the sealing bolt is designed to conform to the connecting tube.

Compared to the related art, the present disclosure has the following beneficial effects. The structure of the present disclosure is scientifically sound, offering both safety and convenience in use.

1. The synchronous expansion mechanism is arranged. Through the coordinated action of the shaft tube, piston, pilot valve, plug, and spring, a pneumatic regulation structure is constructed, achieving more efficient and stable stepless adjustment by utilizing the driving force from the rotating arms, suspension frame, and forceps arm, thereby significantly enhancing the convenience and flexibility of adjusting the oral retractor's expansion dimensions and force. Combined with the synchronous sensing function of the conduit, plug block, air cavity, and finger pressure head, a physician can more intuitively sense and control a supporting force applied to a patient's oral cavity, significantly improving the convenience, intuitiveness, stability, and reliability of regulating oral support for patients.

Supported by pressure readings from the pressure gauge, it assists the physician in precisely regulating an expansion force, thereby ensuring reliable support, avoiding strain on perioral muscles, and enhancing patient comfort. Compatibility with different patients is improved to accommodate varying support requirements. Furthermore, the direct rigid support method of clamp-type oral retractors is replaced with an elastic expansion method for oral support, thereby expanding the adjustable range of expansion while delivering a more compliant and gentle support process. Force distribution across an oral area is optimized, significantly improving both support effectiveness and controllability of the oral retractor. Rehabilitation training requirements are met by addressing distinct force needs during passive stretching training and active strength training, thereby rendering force control operations more convenient and efficient.

2. Through the coordinated action of the insertion tubes, flaring seats, top boxes, bottom boxes, ring plugs, cross tubes, vertical tubes, cylinder bodies, and sealing rings, a bidirectional expansion structure is constructed, enhancing the controllability of the oral retractor's expansion method. On one hand, while ensuring support stability, the oral retractor's expansion method is further optimized to conform to patient-specific oral contours, significantly improving compatibility between the oral retractor and the patient's oral cavity, thereby accommodating variations in oral dimensions and morphological characteristics among different patients, resulting in more flexible, efficient, and patient-friendly support operations.

On the other hand, through the coordinated action of the sliding cylinder, the plug rings, the guide holes, the straight tubes, and the strip boxes, a dual elastic support structure is formed, which can enhance the reliability of the oral retractor's expansion force and the sensitivity of its expansion movements. Additionally, it can adjust angles of the rotating arms, suspension frame, and forceps arm without compromising support force, effectively improving diagnostic visibility, expanding operational space for hand movements, and facilitating more efficient diagnosis and treatment procedures for medical staff.

3. A segmented assembly mechanism is arranged. Through the coordinated action of the valve housings, drive bolts, plug disks, cylindrical cavities, side holes, flow-stop plates, compression springs, and grooves, an elastic connection structure and a rigid locking mechanism are constructed. Combined with the flow-limiting function of the strip plates, the ejector pins, the flower-shaped plates, and the protruding blocks, dynamic connections among the strip boxes, top boxes, bottom boxes, and flaring seats are achieved, allowing for flexible assembly of the oral retractor based on practical requirements, ensuring support integrity while preserving the relative independence of components. Differing patient needs are flexibly accommodated, allowing both independent and integrated support for the lips, cheeks, and teeth. This design not only enhances operational flexibility during oral retractor procedures, achieving more efficient oral dilation for patients and medical staff, but also improves the convenience and reliability of diagnostic, therapeutic, and rehabilitation processes. Furthermore, it facilitates disassembly, replacement, and cleaning procedures for intraoral components, significantly enhancing medical hygiene standards.

Coupled with the synergistic interaction of the lead screws, the toothed plates, the clamping tube, the negative pressure bulb, the squeeze bulb, and the check valves, a dental support structure and a saliva management structure are formed, significantly enhancing the multifunctionality of the oral retractor by integrating oral support and salivary clearance into a single device, thereby improving the stability and reliability of diagnostic and therapeutic procedures. Additionally, through the coordinated action of the connecting tubes, flat boxes, and sealing bolts, a flow-restricting guide structure is constructed, allowing for fine-tuning and calibration of the initial state of the oral retractor, significantly improving the compatibility of the oral retractor's initial dimensions with anatomical variations among patients, thereby facilitating seamless deployment of the support functionality of the oral retractor.

In conclusion, the oral retractor can achieve stepless adjustment of both support force and opening dimensions, along with bidirectional control over its expansion method, thereby ensuring support stability while significantly enhancing the gentleness of the support provided. Patient comfort is enhanced while achieving improved compatibility with the oral contours and dimensions of different patients. Additionally, medical staff can control the expansion force more intuitively, efficiently, and precisely. Combined with a detachable mounting method, the operational flexibility of the oral retractor is further enhanced, accommodating both diagnostic procedures and subsequent rehabilitation training, thereby effectively expanding the scope of application of the oral retractor.

### BRIEF DESCRIPTION OF THE DRAWINGS

The accompanying drawings are provided to further illustrate the present disclosure and form an integral part of the specification. These drawings, in conjunction with the embodiments of the present disclosure, serve to explain the present disclosure without limiting its scope. In the accompanying drawings:

FIG. 1 is a schematic structural diagram of the present disclosure;

FIG. 2 is a schematic diagram of a mounting structure of a flaring seat of the present disclosure;

FIG. 3 is a schematic structural diagram of a synchronous expansion mechanism of the present disclosure;

FIG. 4 is a schematic diagram of a mounting structure of a plug block of the present disclosure;

FIG. 5 is a schematic diagram of a mounting structure of a shaft tube of the present disclosure;

FIG. 6 is a schematic structural diagram of a segmented assembly mechanism of the present disclosure; and

FIG. 7 is a local exploded view of the present disclosure.

Reference numerals and denotations thereof: 1-base body; 2-rotating arm; 3-suspension frame; 4-forceps arm; and 5-cheek plate;

200-synchronous expansion mechanism; 201-shaft tube; 202-piston; 203-pilot valve; 204-conduit; 205-plug block; 206-air cavity; 207-finger pressure head; 208-plug; 209-spring; 210-pressure gauge; 211-sliding cylinder; 212-plug ring; 213-guide hole; 214-straight tube; 215-strip box; 216-insertion tube; 217-flaring seat; 218-top box; 219-bottom box; 220-ring plug; 221-cross tube; 222-vertical tube; 223-cylinder body; and 224-sealing ring;

300-segmented assembly mechanism; 301-valve housing; 302-drive bolt; 303-plug disk; 304-cylindrical cavity; 305-side hole; 306-flow-stop plate; 307-compression spring; 308-groove; 309-strip plate; 310-ejector pin; 311-flower-shaped plate; 312-protruding block; 313-lead screw; 314-toothed plate; 315-clamping tube; 316-negative pressure bulb; 317-squeeze bulb; 318-check valve; 319-connecting tube; 320-flat box; and 321-sealing bolt; and

3091-air valve; and 3141-toothed pad.

### DETAILED DESCRIPTION

The preferred embodiments of the present disclosure are described below in combination with the accompanying drawings. It is to be understood that the preferred embodiments are intended merely to illustrate and explain the present disclosure, rather than limiting the present disclosure.

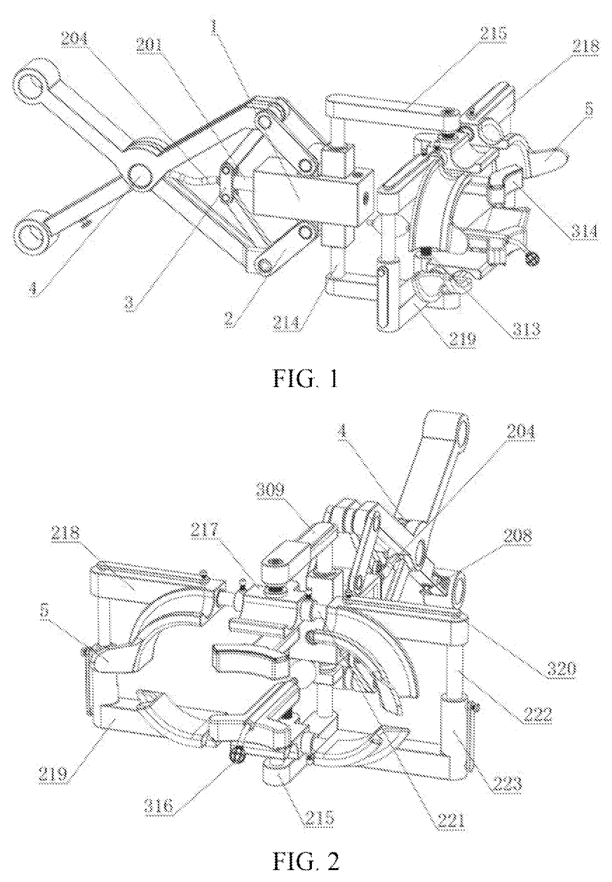

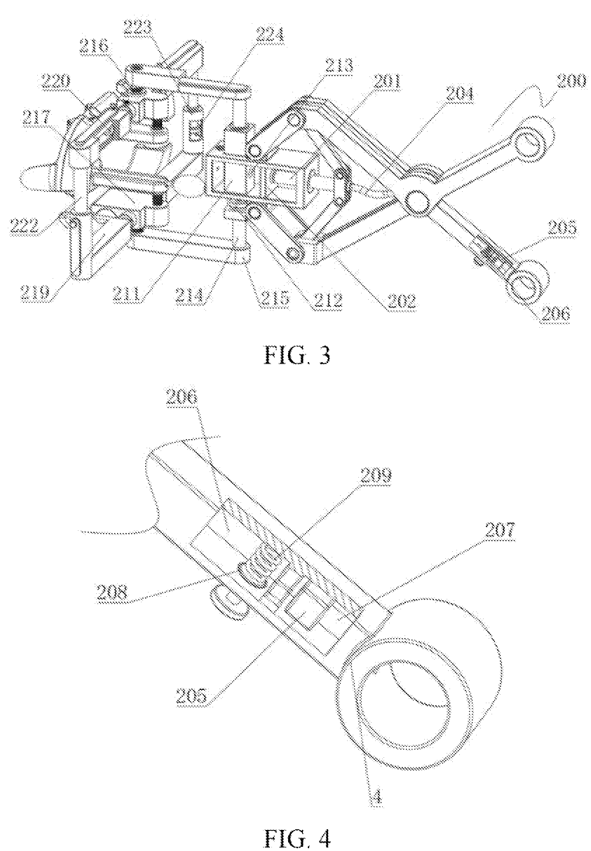

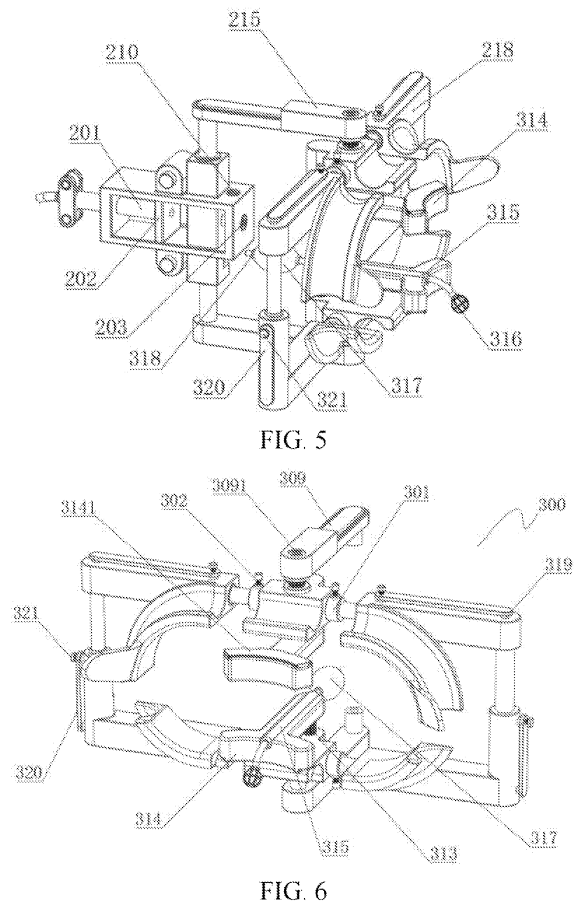

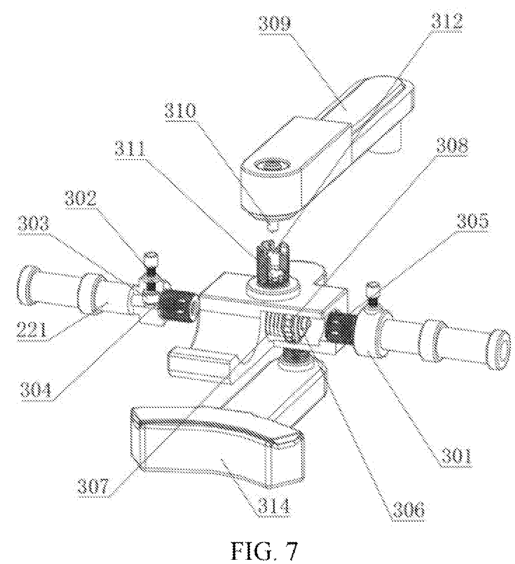

Embodiment: referring to FIGS. 1-7, the present disclosure provides a technical solution: a multifunctional detachable oral retractor for orthodontic treatment includes a base body 1, middle parts of top and bottom ends of the base body 1 are hinged with rotating arms 2, end portions of the rotating arms 2 are hinged with a suspension frame 3, the end portions of the rotating arms 2 are hinged with a forceps arm 4 located on an outer side of the suspension frame 3, and a synchronous expansion mechanism 200 is mounted on an end face of the suspension frame 3.

The synchronous expansion mechanism 200 includes a shaft tube a piston 202, a pilot valve 203, a conduit 204, a plug block 205, an air cavity 206, a finger pressure head 207, a plug 208, a spring 209, a pressure gauge 210, a sliding cylinder 211, plug rings 212, guide holes 213, straight tubes 214, strip boxes 215, insertion tubes 216, flaring seats 217, top boxes 218, bottom boxes 219, ring plugs 220, cross tubes 221, vertical tubes 222, cylinder bodies 223, and sealing rings 224;

the shaft tube 201 is slidably mounted on an end face of one side of the base body 1, the piston 202 is slidably mounted inside the base body 1, the pilot valve 203 is mounted at a middle part of an end face of the other side of the base body 1, the pressure gauge 210 is mounted on one side of the top end of the base body 1, the conduit 204 is mounted at an end portion of the shaft tube 201, the plug block 205 is slidably mounted at one end of the forceps arm 4, the air cavity 206 is disposed inside the forceps arm 4 corresponding to the plug block 205, the finger pressure head 207 is mounted at a middle part of a side end face of the plug block 205, the plug 208 is slidably mounted on a side end face of the air cavity 206 close to the plug block 205, and the spring 209 is mounted at an end portion of the plug 208;

the sliding cylinder 211 is mounted at the top end of the base body 1, the pilot valve 203 is a one-way valve, the air cavity 206 is communicated with the shaft tube 201 through the conduit 204, and the shaft tube 201 and the sliding cylinder 211 are communicated with an inner cavity of the base body 1 through the guide holes 213; the plug 208 has an I-shaped cross-sectional profile, and an original length of the spring 209 is greater than a thickness of the air cavity 206, thereby allowing for stepless adjustment of an expansion force; the straight tubes 214 are slidably mounted at two ends of the sliding cylinder 211, the plug rings 212 are mounted at end portions of the straight tubes 214 located inside the sliding cylinder 211, the guide holes 213 are disposed at middle parts of side end faces of the sliding cylinder 211 and the piston 202, the strip boxes 215 are mounted at the other ends of the straight tubes 214, insertion tubes 216 are threadedly mounted on the other sides of bottom ends of the strip boxes 215, and the flaring seats 217 are rotatably mounted at end portions of the insertion tubes 216;

the top boxes 218 are mounted on two sides of one of the flaring seats 217, the bottom boxes 219 are mounted on two sides of the other flaring seat 217, cheek plates 5 are mounted at corners of arched portions of the top boxes 218, and the arched portions of the top boxes 218 and the bottom boxes 219, along with the cheek plates 5, are designed to conform to a natural curvature of the human oral cavity, thereby enhancing support effectiveness; and the ring plugs 220 are slidably mounted inside the top boxes 218 and the bottom boxes 219, the cross tubes 221 are mounted at end portions of the ring plugs 220, the top boxes 218 and the bottom boxes 219 are sealed and slidably connected to the cross tubes 221, and end portions of the cross tubes 221 are threadedly connected to the flaring seats 217, thereby enhancing support reliability; and

the vertical tube 222 is mounted on one side of a bottom end of each top box 218, and the cylinder body 223 is mounted on one side of a top end of each bottom box 219; interiors of the top boxes 218 and the bottom boxes 219 located on outer sides of the cross tubes 221, and interiors of the cylinder bodies 223 located on outer sides of the vertical tubes 222, are all filled with clean air, thereby enhancing the usability convenience of an oral retractor; and the sealing ring 224 is mounted at an end portion of the vertical tube 222 located inside the cylinder body 223, the sealing ring 224 is slidably connected to the cylinder body 223, the cylinder body 223 is designed to conform to a shape of the sealing ring 224, and a sliding distance of the sealing ring 224 is equal to that of the plug ring 212, thereby enhancing expansion stability.

A segmented assembly mechanism 300 includes valve housings 301, drive bolts 302, plug disks 303, cylindrical cavities 304, side holes 305, flow-stop plates 306, compression springs 307, grooves 308, strip plates 309, ejector pins 310, flower-shaped plates 311, protruding blocks 312, lead screws 313, toothed plates 314, a clamping tube 315, a negative pressure bulb 316, a squeeze bulb 317, check valves 318, connecting tubes 319, flat boxes 320, and sealing bolts 321;

the valve housings 301 are mounted on outer curved faces of the cross tubes 221 close to the flaring seats 217, and the drive bolts 302 are threadedly mounted at tops of outer curved faces of the valve housings 301; the plug disk 303 is mounted at an end portion of each drive bolt 302, and the cylindrical cavity 304 is disposed inside each cross tube 221 corresponding to the plug disk 303; and a plurality of side holes 305 are disposed circumferentially at equal angles on the outer curved faces of the cross tubes 221 located inside each flaring seat 217, flow-stop plates 306 are slidably mounted inside the flaring seat 217 in a symmetrical manner, a compression spring 307 is mounted at a middle part of side end faces of the flow-stop plates 306, and a plurality of grooves 308 are disposed circumferentially at equal angles on an outer curved face of each flow-stop plate 306;

the strip plate 309 is slidably mounted inside each strip box 215, an ejector pin 310 is mounted on an end face of the strip plate 309 corresponding to the insertion tube 216, and a flower-shaped plate 311 is slidably mounted inside the insertion tube 216; an original length of the compression spring 307 exceeds twice a sliding distance of the flow-stop plate 306, a minimum clearance between the flow-stop plate 306 and the end face of the flaring seat 217 is less than a distance between the valve housing 301 and the side holes 305, a clearance between the strip plate 309 and a bottom end of the strip box 215 is less than a length of the insertion tube 216, and a minimum clearance between the flower-shaped plate 311 and the end portion of the insertion tube 216 is less than a length of the ejector pin 310; an air valve 3091 is mounted on an end face of the strip box 215 away from one side corresponding to a length of the insertion tube 216, and an interior of the strip box 215 located between the air valve 3091 and the strip plate 309 is filled with clean air, thereby enhancing the operational flexibility of the oral retractor; and a plurality of protruding blocks 312 are mounted circumferentially at equal angles at an end portion of the insertion tube 216, the lead screws 313 are threadedly mounted on end faces of the flaring seats 217 close to one side of the base body 1, the toothed plates 314 are mounted at end portions of the lead screws 313, and the clamping tube 315 is clamped into a middle part of a top end of one of the toothed plates 314;

the negative pressure bulb 316 is mounted at one end of the clamping tube 315, the squeeze bulb 317 is mounted at the other end of the negative pressure bulb 316, and the check valves 318 are mounted at two ends of the squeeze bulb 317 corresponding to the clamping tube 315; a toothed pad 3141 is adhesively bonded to an end face of each toothed plate 314, and a cross-sectional profile of the toothed pad 3141 is designed to conform to a dental arch curvature of the human oral cavity; and the check valves 318 at the two ends of the squeeze bulb 317 are an inlet one-way valve and an outlet one-way valve, the squeeze bulb 317 is connected to the clamping tube 315 via the inlet one-way valve, and the negative pressure bulb 316 is a hollow mesh structure for saliva management; and

the connecting tubes 319 are mounted on two sides of top ends of the top boxes 218, two sides of bottom ends of the bottom boxes 219, and two sides of outer curved faces of each cylinder body 223; flat boxes 320 are mounted at end portions of the connecting tubes 319, and sealing bolts 321 are threadedly mounted on end faces of the flat boxes 320 corresponding to the connecting tubes 319; spaces on two sides of each ring plug 220 within an inner cavity of the top box 218 or the bottom box 219, are interconnected via the connecting tube 319 and the flat box 320; spaces on two sides of each sealing ring 224 within an inner cavity of the cylinder body 223, are interconnected via the connecting tube 319 and the flat box 320; and a length of the sealing bolt 321 exceeds a thickness of the flat box 320, and an end portion of the sealing bolt 321 is designed to conform to the connecting tube 319, thereby allowing for fine-tuning and calibration of an initial state of the oral retractor.

An operating principle and a usage flow of the present disclosure are as follows. This multifunctional detachable oral retractor for orthodontic treatment requires selective assembly of its corresponding components based on clinical requirements before practical use. The device is designed to provide localized and integrated support for the patient's lips, teeth, and cheeks while performing salivary adsorption and clearance. This design accommodates different needs for diagnostic visualization and rehabilitation training. The following operational description will focus on the integrated support as the primary example. Adaptation to varying patient needs can be achieved by removing corresponding components from this integrated support configuration.

A main unit including the base body 1, the rotating arms 2, suspension frame 3, and the forceps arm 4 is extracted, and the flaring seat 217 is extracted with its insertion tube 216 aligned with an opening on the strip box 215. The insertion tube 216 is rotated, and the insertion tube 216 is threaded into the strip box 215. The two flaring seats 217 are sequentially secured to the strip boxes 215 to establish stable connections. During this process, as the insertion tube 216 is threaded into the strip box 215, it pushes against the strip plate 309, forcing the strip plate 309 to compress the air on the other side and slide within the strip box 215. Simultaneously, the ejector pin 310 pushes against the flower-shaped plate 311, forcing the flower-shaped plate 311 to slide within the insertion tube 216, thereby establishing an airflow passage between the strip box 215 and the flaring seat 217. It is to be noted that the air pressure between the strip plate 309 and the air valve 3091 inside the strip box 215 needs to exceed the pressure on the other side of the strip plate 309 before use, thereby ensuring the mounting stability of the flaring seat 217.

Subsequently, the cross tubes 221 on the top boxes 218 are aligned with an opening of the upper flaring seat 217, and the cross tubes 221 on the bottom boxes 219 are aligned with an opening of the lower flaring seat 217. The cross tubes 221 are rotated to mount the top boxes 218 and the bottom boxes 219 onto the corresponding flaring seats 217. Alternatively, the mounting orientation may be reversed according to personal preference for gripping the forceps arm 4, provided that the two top boxes 218 are simultaneously mounted on one flaring seat 217 and the two bottom boxes 219 are mounted on the other flaring seat 217. During the threading process of the cross tubes 221 into the flaring seat 217, the cross tubes 221 press against the flow-stop plates 306, forcing the two flow-stop plates 306 to overcome the spring force of the compression spring 307 and move closer together, thereby establishing communication between the side holes 305 and an inner cavity of the flaring seat 217. Subsequently, the drive bolts 302 are rotated, driving the plug disks 303 to slide along the cylindrical cavities 304, thereby opening airflow passages from the inner cavities of both the top boxes 218 and the bottom boxes 219 to the two flaring seats 217.

The toothed plate 314 is extracted with the lead screw 313 aligned with a hole on the flaring seat 217, and the lead screw 313 is rotated to thread it into the flaring seat 217. The toothed plate 314, to which the clamping tube 315 can be mounted, is mounted onto the lower flaring seat 217, and the other toothed plate 314 is mounted onto the upper flaring seat 217. Furthermore, the clamping tube 315 is retrieved, and the clamping tube 315 is clamped to the lower toothed plate 314. By adjusting a threading depth of the lead screw 313 into the flaring seat 217, a distance between the toothed plate 314 and the flaring seat 217 can be precisely regulated to accommodate individual patient variations. The toothed pads 3141 are attached to recessed areas of the toothed plates 314 to complete the full assembly of the oral retractor. Only necessary adjustments need to be made before initial use before it can be officially put into service.

The sealing bolts 321 are rotated to unthread the end portions from the connecting tubes 319, thereby opening flow passages of the connecting tubes 319. The forceps arm 4 is gripped, and the suspension frame 3 drives the shaft tube 201 in reciprocating displacement under the constraint of the rotating arms 2, forcing the piston 202 to slide reciprocally within the base body 1 under the drive of the shaft tube 201. Subsequently, under the flow restriction of the pilot valve 203, external air is drawn into the base body 1, compressed through the guide holes 213 into the sliding cylinder 211. The airflow passes through the straight tubes 214 into the strip boxes 215, proceeds via the insertion tubes 216 into the flaring seats 217, enters the top boxes 218 and the bottom boxes 219 through the cross tubes 221, and flows through the vertical tubes 222 into the cylinder bodies 223. For descriptive convenience in the subsequent sections, this airflow path is hereby designated as a pneumatic pathway. Directed by the connecting tubes 319 and the flat boxes 320, the airflow enters the spaces within the top boxes 218 and the bottom boxes 219 located on the outer sides of the cross tubes 221, as well as the spaces within the cylinder bodies 223 located on the outer sides of the vertical tubes 222.

In an initial state, the spaces within the top boxes 218 and the bottom boxes 219 located on the outer sides of the cross tubes 221, as well as the spaces within the cylinder bodies 223 located on the outer sides of the vertical tubes 222, are all filled with air to maintain the flaring seats 217, the top boxes 218, the bottom boxes 219, and the toothed plates 314 into a mutually approximated and retracted state, thereby facilitating insertion of the oral retractor into the patient's oral cavity.

The above adjustments are required only before the initial use on the patient, serving to fine-tune the spacing among the flaring seats 217, top boxes 218, bottom boxes 219, and toothed plates 314 in the initial state, and to adjust overall initial expansion dimensions of the oral retractor to accommodate the patient's oral cavity. Upon completion of the above adjustments, the sealing bolts 321 are rotated counterclockwise to block the flow passages in the connecting tubes 319, thereby locking the initial configuration. This ensures that the oral retractor may automatically return to its initial state under pneumatic pressure when external support is removed, after which the device is officially put into service.

During the use of the oral retractor, the forceps arm 4 is first gripped. In this process, two fingers need to be inserted into holes at end portions of the forceps arm 4, with one finger aligned with and positioned to readily depress the plug 208. The gripping manner of the forceps arm 4 may be adaptively adjusted according to operator preference. The plug 208 is depressed, causing air within the air cavity 206 to discharge through a clearance between the plug 208 and the air cavity 206. Concurrently, air within the base body 1 is discharged through the communication established by the shaft tube 201, the conduit 204, and the guide holes 213. Accordingly, air previously charged into the top boxes 218, the bottom boxes 219, and the cylinder bodies 223 via the above pneumatic pathway is subsequently discharged.

Due to the flow passages of the connecting tubes 319 being blocked, the air from the spaces within the top boxes 218 and the bottom boxes 219 located on the outer sides of the cross tubes 221, as well as the spaces within the cylinder bodies 223 located on the outer sides of the vertical tubes 222, is retained. At this time, a pressure differential is created between the ring plugs 220 and the sealing rings 224, thereby causing the oral retractor to return to its initial state under pneumatic pressure.

Upon being prevented from depression, the plug 208 is returned to its initial position under the drive of the spring 209, thereby resealing the clearance between the air cavity 206 and the plug 208. After the arched portions of flaring seats 217 are sent into the patient's oral cavity, and the patient is allowed to bite the toothed pads 3141 on the toothed plates 314, reciprocating pressure is reapplied to the forceps arm 4 to evacuate external air. This air is introduced through the above pneumatic pathway into the top boxes 218, the bottom boxes 219, and the cylinder bodies 223, forcing the ring plugs 220 and the sealing rings 224 to slide against the pressure on the other side via pneumatic actuation, thereby progressively increasing the spacing among the flaring seats 217, the top boxes 218, the bottom boxes 219 and the toothed plates 314, such that the overall dimensions of the oral retractor are expanded outward.

During this process, the portions of the flaring seats 217, the top boxes 218, and the bottom boxes 219 that are inserted into the patient’s oral cavity provide elastic support to the patient’s lips, the cheek plates 5 provide elastic support to the patient’s cheeks, and the toothed plates 314 provide elastic support to the patient’s teeth, thereby propping the patient’s mouth open during the overall expansion of the oral retractor.

Through the communication of the guide holes 213, the shaft tube 201, and the conduit 204, air drawn into the base body 1 is simultaneously delivered into the air cavity 206, forcing the plug block 205 to push the finger pressure head 207 against a physician's finger under air pressure. Under identical pressure conditions, the plug block 205 drives the finger pressure head 207 to press against the physician's finger, assisting the physician in more intuitively perceiving a support force exerted on the patient's oral cavity. Coupled with readings from the pressure gauge 210, the support force applied to the patient's oral cavity is precisely controlled and adjusted steplessly according to actual requirements, thereby adapting to various oral dimensions while preventing strain to the perioral muscles of the patient.

The above process constitutes the procedure for propping open the patient's mouth and providing comprehensive support. The above mounting and adjustment operations are required only during initial use. Subsequently, the mouth can be propped open simply by squeezing the forceps arms 4 to inflate the top boxes 218, the bottom boxes 219, and the cylinder bodies 223, and by pressing the plug 208 to release air. The support force applied to the oral cavity and the extent to which the patient's mouth is propped open can be controlled through regulation of the air pressure.

During the observation and diagnosis by the physician, if it is only necessary to provide support to the patient's teeth and prop open the patient's mouth, the top boxes 218 and the bottom boxes 219 may be omitted during the above assembly stage. The patient's mouth is propped open solely by the flaring seats 217 and the toothed plates 314, in which the strip boxes 215 are rotatably connected to the flaring seats 217 via the insertion tubes 216. During this process, the base body 1, the rotating arms 2, the suspension frame 3, and the forceps arm 4 may be pivoted to one side, thereby improving visibility and facilitating observation. Similarly, during rehabilitation training, if only support to the patient's teeth is required, the top boxes18 and the bottom boxes 19 may also be omitted. By regulating the air pressure, the support force applied to the oral cavity can be adjusted steplessly, thereby meeting different force requirements during both passive stretching training and active strength training.

Conversely, if fully propping open the patient's lips is required during diagnostic or therapeutic procedures, the top boxes 218 and the bottom boxes 219 may be mounted during the above assembly stage. Furthermore, to further enhance the visual and operational space, upon completion of the air pressure adjustment, the insertion tubes 216 may be rotated in the reverse direction to disengage the strip boxes 215 from the top boxes 218 and the bottom boxes 219, allowing for the removal of the base body 1, the rotating arms 2, the suspension frame 3, the forceps arms 4, and the strip boxes 215, thereby expanding both the visual field and the space for manual operation.

When saliva removal is required, the squeeze bulb 317 is squeezed repeatedly to create a negative pressure at the negative pressure bulb 316 under the guidance and regulation of the check valves 318, forcing saliva to be filtered through the negative pressure bulb 316 and enter the clamping tube 315. The filtered saliva passes through the inlet one-way valve within the check valve 318 to enter the squeeze bulb 317, and is subsequently discharged through the outlet one-way valve within the check valve 318. During the assembly stage, an external fluid collection bag may be attached to an end portion of the outlet one-way valve within the check valve 318 if saliva removal is required. If saliva removal is not required, the clamping tube 315 may be omitted during assembly.

During disassembly and assembly, in the absence of the supporting force provided by the insertion tubes 216, the strip plates 309 may conform to the strip boxes 215 under air pressure action, thereby connecting communication ports between the strip boxes 215 and the insertion tubes 216. In the absence of the supporting force provided by the cross tubes 221, the flow-stop plates 306, under the action of the compression spring 307, conform to the openings on two sides of the flaring seat 217. Combined with the on-off function of the plug disks 303 and the cylindrical cavities 304, this configuration maintains internal airflow patency during assembly of the various components while ensuring the relative independence of the internal air medium within each component during disassembly. As a result, the oral retractor is capable of retaining elastic support force even in a partially assembled state.

Finally, it is to be noted that the foregoing is only the preferred embodiment of the present disclosure and is not intended to limit the present disclosure. Although the present disclosure has been described by the above embodiments in detail, a person skilled in the art may still modify the technical solutions described in the above embodiments, or perform equivalent substitutions for some technical features thereof. Any modifications, equivalent substitutions, improvements, and the like made within the spirit and principles of the present disclosure are included within the scope of protection of the present disclosure.

1. A multifunctional detachable oral retractor for orthodontic treatment, comprising a base body (1), wherein middle parts of top and bottom ends of the base body (1) are hinged with rotating arms (2), end portions of the rotating arms (2) are hinged with a suspension frame (3), the end portions of the rotating arms (2) are hinged with a forceps arm (4) located on an outer side of the suspension frame (3), and a synchronous expansion mechanism (200) is mounted on an end face of the suspension frame (3);
the synchronous expansion mechanism (200) comprises a shaft tube (201);
the shaft tube (201) is slidably mounted on an end face of one side of the base body (1), a piston (202) is slidably mounted inside the base body (1), a pilot valve (203) is mounted at a middle part of an end face of the other side of the base body (1), a pressure gauge (210) is mounted on one side of the top end of the base body (1), a conduit (204) is mounted at an end portion of the shaft tube (201), a plug block (205) is slidably mounted at one end of the forceps arm (4), an air cavity (206) is disposed inside the forceps arm (4) corresponding to the plug block (205), a finger pressure head (207) is mounted at a middle part of a side end face of the plug block (205), a plug (208) is slidably mounted on a side end face of the air cavity (206) close to the plug block (205), and a spring (209) is mounted at an end portion of the plug (208);
a sliding cylinder (211) is mounted at the top end of the base body (1), straight tubes (214) are slidably mounted at two ends of the sliding cylinder (211), plug rings (212) are mounted at end portions of the straight tubes (214) located inside the sliding cylinder (211), guide holes (213) are disposed at middle parts of side end faces of the sliding cylinder (211) and the piston (202), strip boxes (215) are mounted at the other ends of the straight tubes (214), insertion tubes (216) are threadedly mounted on the other sides of bottom ends of the strip boxes (215), and flaring seats (217) are rotatably mounted at end portions of the insertion tubes (216);
the pilot valve (203) is a one-way valve, the air cavity (206) is communicated with the shaft tube (201) through the conduit (204), and the shaft tube (201) and the sliding cylinder (211) are communicated with an inner cavity of the base body (1) through the guide holes (213);
top boxes (218) are mounted on two sides of one of the flaring seats (217), bottom boxes (219) are mounted on two sides of the other flaring seat (217), ring plugs (220) are slidably mounted inside the top boxes (218) and the bottom boxes (219), cross tubes (221) are mounted at end portions of the ring plugs (220), a vertical tube (222) is mounted on one side of a bottom end of each top box (218), a cylinder body (223) is mounted on one side of a top end of each bottom box (219), and a sealing ring (224) is mounted at an end portion of the vertical tube (222) located inside the cylinder body (223);
the top boxes (218) and the bottom boxes (219) are sealed and slidably connected to the cross tubes (221); and
through the communication of the guide holes (213), the shaft tube (201), and the conduit (204), air drawn into the base body (1) is simultaneously delivered into the air cavity (206), forcing the plug block (205) to push the finger pressure head (207) against a physician's finger under air pressure; under identical pressure conditions, the plug block (205) drives the finger pressure head (207) to press against the physician's finger, assisting the physician in more intuitively perceiving a support force exerted on a patient's oral cavity; and coupled with readings from the pressure gauge (210), the support force applied to the patient's oral cavity is precisely controlled and adjusted steplessly according to actual requirements.

2. The multifunctional detachable oral retractor for orthodontic treatment according to claim 1, wherein the plug (208) has an I-shaped cross-sectional profile, and an original length of the spring (209) is greater than a thickness of the air cavity (206).

3. The multifunctional detachable oral retractor for orthodontic treatment according to claim 1, wherein cheek plates (5) are mounted at corners of arched portions of the top boxes (218), and the arched portions of the top boxes (218) and the bottom boxes (219), along with the cheek plates (5), are designed to conform to a natural curvature of the human oral cavity.

4. The multifunctional detachable oral retractor for orthodontic treatment according to claim 1, wherein end portions of the cross tubes (221) are threadedly connected to the flaring seats (217).

5. The multifunctional detachable oral retractor for orthodontic treatment according to claim 1, wherein interiors of the top boxes (218) and the bottom boxes (219) located on outer sides of the cross tubes (221), and interiors of the cylinder bodies (223) located on outer sides of the vertical tubes (222), are all filled with clean air.

6. The multifunctional detachable oral retractor for orthodontic treatment according to claim 1, wherein the sealing ring (224) is slidably connected to the cylinder body (223), the cylinder body (223) is designed to conform to a shape of the sealing ring (224), and a sliding distance of the sealing ring (224) is equal to that of the plug ring (212).

7. The multifunctional detachable oral retractor for orthodontic treatment according to claim 1, wherein valve housings (301) are mounted on outer curved faces of the cross tubes (221) close to the flaring seats (217), and drive bolts (302) are threadedly mounted at tops of outer curved faces of the valve housings (301); a plug disk (303) is mounted at an end portion of each drive bolt (302), and a cylindrical cavity (304) is disposed inside each cross tube (221) corresponding to the plug disk (303); and a plurality of side holes (305) are disposed circumferentially at equal angles on the outer curved faces of the cross tubes (221) located inside each flaring seat (217), flow-stop plates (306) are slidably mounted inside the flaring seat (217) in a symmetrical manner, a compression spring (307) is mounted at a middle part of side end faces of the flow-stop plates (306), and a plurality of grooves (308) are disposed circumferentially at equal angles on an outer curved face of each flow-stop plate (306);
a strip plate (309) is slidably mounted inside each strip box (215), an ejector pin (310) is mounted on an end face of the strip plate (309) corresponding to the insertion tube (216), a flower-shaped plate (311) is slidably mounted inside the insertion tube (216), and a plurality of protruding blocks (312) are mounted circumferentially at equal angles at an end portion of the insertion tube (216); and lead screws (313) are threadedly mounted on end faces of the flaring seats (217) close to one side of the base body (1), toothed plates (314) are mounted at end portions of the lead screws (313), and a clamping tube (315) is clamped into a middle part of a top end of one of the toothed plates (314); and
a negative pressure bulb (316) is mounted at one end of the clamping tube (315), a squeeze bulb (317) is mounted at the other end of the negative pressure bulb (316), and check valves (318) are mounted at two ends of the squeeze bulb (317) corresponding to the clamping tube (315); and connecting tubes (319) are mounted on two sides of top ends of the top boxes (218), two sides of bottom ends of the bottom boxes (219), and two sides of outer curved faces of each cylinder body (223); and flat boxes (320) are mounted at end portions of the connecting tubes (319), and sealing bolts (321) are threadedly mounted on end faces of the flat boxes (320) corresponding to the connecting tubes (319).

8. The multifunctional detachable oral retractor for orthodontic treatment according to claim 7, wherein an original length of the compression spring (307) exceeds twice a sliding distance of the flow-stop plate (306), a minimum clearance between the flow-stop plate (306) and the end face of the flaring seat (217) is less than a distance between the valve housing (301) and the side holes (305), a clearance between the strip plate (309) and a bottom end of the strip box (215) is less than a length of the insertion tube (216), and a minimum clearance between the flower-shaped plate (311) and the end portion of the insertion tube (216) is less than a length of the ejector pin (310); and an air valve (3091) is mounted on an end face of the strip box (215) away from one side corresponding to a length of the insertion tube (216), and an interior of the strip box (215) located between the air valve (3091) and the strip plate (309) is filled with clean air.

9. The multifunctional detachable oral retractor for orthodontic treatment according to claim 7, wherein a toothed pad (3141) is adhesively bonded to an end face of each toothed plate (314), and a cross-sectional profile of the toothed pad (3141) is designed to conform to a dental arch curvature of the human oral cavity; and the check valves (318) at the two ends of the squeeze bulb (317) are an inlet one-way valve and an outlet one-way valve, the squeeze bulb (317) is connected to the clamping tube (315) via the inlet one-way valve, and the negative pressure bulb (316) is a hollow mesh structure.

10. The multifunctional detachable oral retractor for orthodontic treatment according to claim 7, wherein spaces on two sides of each ring plug (220) within an inner cavity of the top box (218) or the bottom box (219) are interconnected via the connecting tube (319) and the flat box (320); spaces on two sides of each sealing ring (224) within an inner cavity of the cylinder body (223) are interconnected via the connecting tube (319) and the flat box (320); and a length of the sealing bolt (321) exceeds a thickness of the flat box (320), and an end portion of the sealing bolt (321) is designed to conform to the connecting tube (319).
